(19) United States
(12) Patent Application Publication
(10) Pub. No.: US 2022/0260568 A1
(43) Pub. Date: Aug. 18, 2022

(54) MEMBRANE-TYPE SURFACE-STRESS SENSOR AND ANALYSIS METHOD USING THE SAME

(71) Applicant: NEC Corporation, Minato-ku, Tokyo (JP)

(72) Inventor: Kenji MIYAZAKI, Minato-ku, Tokya (JP)

(73) Assignee: NEC Corporation, Minato-ku, Tokyo (JP)

(21) Appl. No.: 17/625,388

(22) PCT Filed: Jun. 19, 2020
(86) PCT No.: PCT/JP2020/024068

§ 371 Date: Jan. 7, 2022

(30) Foreign Application Priority Data
Jul. 10, 2019 (JP) 2019-128311
Mar. 26, 2020 (JP) 2020-056897

(51) Int. Cl. G01N 33/573; B01L 3/00; G01N 27/12

(52) U.S. Cl. CPC G01N 33/573; B01L 3/502715; B01L 2300/0645; G01N 27/125; B01L 3/502761

(57) ABSTRACT

The present invention provides a membrane-type surface-stress sensor which has a new form for binding a target. A membrane-type surface-stress sensor of the present invention includes: aptamers; a membrane; and a sensor substrate, wherein the aptamer is a nucleic acid molecule that binds to a target and is immobilized to the membrane, the membrane is a membrane that deforms upon binding of the target to the aptamer, the sensor substrate has a support region, the support region supports the membrane and has a piezoresistive element, and the piezoresistive element is an element for detecting deformation of the membrane.

### TECHNICAL FIELD

The present invention relates to a membrane-type surface-stress sensor and an analysis method using the same.

### BACKGROUND ART

In a wide variety of fields such as food, medicine, and the like, detection of targets is important, and various methods have been proposed. In recent years, a membrane-type surface-stress sensor has attracted attention (see Patent Literature 1). The membrane-type surface-stress sensor can analyze the presence or absence or the amount of a target by, for example, binding a target to a membrane such as a silicon membrane, deforming the membrane, and measuring a variation in electric resistance due to the deformation. However, there is a demand for further improvement of the method for binding a target to the membrane from the viewpoint of, for example, improvement of analysis accuracy and expansion of target to be applied.

### CITATION LIST

### Patent Literature

Patent Literature 1: WO2011/148774

### SUMMARY OF INVENTION

### Technical Problem

With the foregoing in mind, it is an object of the present invention to provide a membrane-type surface-stress sensor which has a new form for binding a target.

### Solution to Problem

In order to achieve the above object, the present invention provides a membrane-type surface-stress sensor, including: aptamers; a membrane; and a sensor substrate, wherein the aptamer is a nucleic acid molecule that binds to a target and is immobilized to the membrane, the membrane is a membrane that deforms upon binding of the target to the aptamer, the sensor substrate has a support region, the support region supports the membrane and has a piezoresistive element, and the piezoresistive element is an element for detecting deformation of the membrane.

The present invention also provides a method for analyzing a target, including the steps of: immersing the membrane-type surface-stress sensor according to the present invention in a sample liquid; applying a voltage to the membrane-type surface-stress sensor in a liquid phase; and analyzing a target in the sample liquid by measuring a stress change of the piezoresistive element in the membrane-type surface-stress sensor.

### Advantageous Effects of Invention

According to the present invention, as a new form of binding a target, by immobilizing aptamers to the membrane, the possibilities such as application to a target different from that of a conventional membrane-type surface-stress sensor and modification different from that of a conventional membrane-type surface-stress sensor can be expanded, for example.

### BRIEF DESCRIPTION OF DRAWINGS

FIG. 1 is a schematic diagram showing a general configuration of the MSS.

FIG. 2 shows a schematic diagram showing the structure of the MSS in Example 2 and graphs showing the voltage of the MSS.

### DESCRIPTION OF EXAMPLE EMBODIMENTS

In the present invention, hereinafter, a “membrane-type surface-stress sensor” is also referred to as a MSS. In a so-called MSS, a membrane having a binding property to a target is supported by a support having a piezoresistive element. When the target binds to the membrane, the membrane suffers stress due to the binding, and the membrane deforms due to the generation of distortion or the like (generation of distortion). A stress is generated in the piezoresistive element of the support that supports the membrane depending on the amount of deformation of the membrane, and the resistance value of the piezoresistive element changes in proportion to the stress. Therefore, by applying a voltage to the MSS and measuring an electric signal accompanying a change in resistance value, it is possible to indirectly analyze the presence or absence of the target bonded to the membrane or to analyze the amount of the target bonded to the membrane. The present invention is characterized in that an aptamer that binds to a target is used in such a MSS, specifically, the aptamer is immobilized to the membrane to bind the target to the MSS. Therefore, other than immobilizing aptamers to the membrane, the configuration of the present invention is not particularly limited, and existing configurations and configurations in future that exhibit similar functions can be utilized.

In the present invention, an “aptamer” is a nucleic acid molecule having a binding property to a target. The aptamer can also be, for example, a nucleic acid molecule that specifically binds to a target. Examples of the constituent unit of the aptamer include nucleotide residues and non-nucleotide residues. Examples of the nucleotide residue include deoxyribonucleotide residues and ribonucleotide residues, wherein the nucleotide residues may be modified or unmodified, for example. Examples of the aptamer include DNA aptamers consisting of deoxyribonucleotide residues, RNA aptamers consisting of ribonucleotide residues, aptamers including both deoxyribonucleotide residues and ribonucleotide residues, and aptamers including modified nucleotide residues. The length of the aptamer is not particularly limited and is, for example, from 10 to 200 bases. For example, existing aptamers may be used as the aptamer to the target, or aptamers newly obtained by a SELEX method or the like may be used depending on the target, for example.

In the present invention, the “target” is not particularly limited and may be any substance capable of contacting the aptamer in a liquid, for example. Examples of the target include microorganisms including bacteria such as anthrax, Escherichia coli, Salmonella, Escherichia coli, and the like; viruses such as influenza virus, and the like; and allergens. Examples of the allergen include grains such as wheat, and the like; eggs; meat; fish; shellfish; vegetables; fruits; milk; beans such as peanuts, and the like; and pollens such as cedar pollen, cypress pollen, and the like. The type of the target is not particularly limited, and examples thereof include polymer compounds such as a protein, a sugar chain, a nucleic acid, a polymer, and the like; and low-molecular compounds.

In the present invention, the “liquid sample” is not particularly limited as long as it is a liquid. When a collected sample is a liquid, the collected sample may be used as a liquid sample as it is or after diluting, suspending, dispersing, or the like with a liquid solvent liquid. When a collected sample is a solid, for example, a liquid sample may be prepared by dissolving, suspending, dispersing, or the like with a liquid solvent. In addition, when a collected sample is a gas, for example, a liquid sample may be prepared by concentrating an aerosol in the gas or may be prepared by further dissolving, suspending, dispersing, or the like with a liquid solvent. The type of the liquid solvent is not particularly limited, and is, for example, a solvent which hardly affects the binding between an aptamer and a target, and specific examples of the liquid solvent include water and a buffer. Examples of the collected sample include food, blood, urine, saliva, body fluid, soil, drainage, tap water, pond, river, and air.

Example embodiments of the present invention will be described. Note here that the present invention is not limited to the following example embodiments. In addition, the descriptions of the respective example embodiments can be referred to each other unless otherwise specified. Furthermore, the configurations of the example embodiments can be combined unless otherwise specified.

### First Example Embodiment

A MSS of the present example embodiment is, as described above, characterized in that it includes aptamers; a membrane; and a sensor substrate, wherein the aptamer is a nucleic acid molecule that binds to a target and is immobilized to the membrane, the membrane is a membrane that deforms upon binding of the target to the aptamer, the sensor substrate has a support region, the support region supports the membrane and has a piezoresistive element, and the piezoresistive element is an element for detecting deformation of the membrane.

In the MSS of the present example embodiment, the membrane is also referred to as a MSS membrane. As described above, the MSS membrane is not particularly limited as long as it is deformed by the binding of the target to the aptamer and the deformation gives a stress to the piezoresistive element. The membrane is, for example, a thin membrane, and the thickness and the area of each surface thereof are not particularly limited, and are same as, for example, a MSS membrane used in a commercially available MSS. The planar shape of the membrane is, for example, a circle, and specifically, is, for example, a regular circle. The material of the membrane is not particularly limited, and is, for example, a silicon membrane, and a specific example is n-type Si(100).

In the MSS of the present example embodiment, the aptamers are immobilized to the MSS membrane. The aptamers may be immobilized to one surface of the MSS membrane or may be immobilized to both surfaces of the MSS membrane, for example. When the aptamers are immobilized to both surfaces of the MSS membrane, it is preferable that the aptamer on one surface and the aptamer on the other surface be, for example, the same aptamer that binds to the same target. In the following description, the surface of the MSS membrane may be, for example, one surface or both surfaces.

The method of immobilizing the aptamers to the MSS membrane is not particularly limited, and the aptamers may be directly immobilized or indirectly immobilized to the MSS membrane. In the former case, for example, by chemically treating the MSS membrane and the aptamers, the aptamers can be immobilized to the MSS membrane by covalent binding or the like. The direct immobilization method may be, for example, a method of utilizing photolithography, and specifically, reference can be made to the specification or the like of U.S. Pat. No. 5,424,186. Another direct immobilization method may be, for example, a method of synthesizing the sensor on the MSS membrane. This method may be, for example, a so-called spot method, and specifically, reference can be made to the specification or the like of U.S. Pat. No. 5,807,522. In the latter case, for example, the aptamer can be immobilized to the MSS membrane via a linker. The type of the linker is not limited in any way, and may be the combination of biotin or a biotin derivative (hereinafter, collectively referred to as biotin) and avidin or an avidin derivative (hereinafter, collectively referred to as avidin). The biotin derivatives include, for example, biocytin and the like, and the avidin derivatives include, for example, streptavidin and the like. The length of the linker may be expressed, for example, by a length of a shortest molecular chain (main chain length) from a functional group on a MSS membrane (e.g., an oxygen atom of a silanol group on a silicon membrane) to an affinity tag such as avidin or an aptamer. The main chain length of the linker is 1 to 20 and is preferably 1 to 15, 1 to 13, 3 to 13, 5 to 13, 1 to 11, 3 to 11, 1 to 10, 3 to 10, 1 to 8, 3 to 8, 1 to 5, 1 to 3, 1 or 2 because the sensitivity of the MSS can be improved. Hereinafter, an immobilization method will be exemplified, but the present invention is not limited thereto.

As a first example, the biotin is bonded to one of the MSS membrane and the aptamer, and avidin is bonded to the other. Then, by binding the biotin with the avidin, the aptamer can be indirectly immobilized to the MSS membrane.

Note that, in the first example, the aptamer is indirectly immobilized to the MSS membrane by utilizing the avidin-biotin specific binding, i.e., the affinity of the biotin to the avidin, but the present invention is not limited thereto, and an affinity tag other than the avidin-biotin may be utilized. As the affinity tag, for example, His tag (His×6 tag)-nickel ion, glutathione-S-transferase-glutathione, maltose binding protein-maltose, epitope tag (myc tag, FLAG tag, HA (hemagglutinin) tag)-antibody or antigen-binding fragment can be utilized. Also in the second to fourth examples described below, affinity tags other than the avidin-biotin may be used.

As a second example, the aptamer may be immobilized to the MSS membrane, e.g., via an intervening membrane. The intervening membrane is formed on the MSS membrane, and as in the first example, the biotin is bonded to one of the intervening membrane and the aptamer, and the avidin is bonded to the other, and then the biotin is bonded with the avidin, thereby immobilizing the aptamer to the MSS via the intervening membrane. The intervening membrane is, for example, a membrane of a metal such as gold, and can be formed by depositing the metal with respect to the MSS membrane. The thickness of the intervening membrane is not particularly limited, and is, for example, 10 to 100 nm. The intervening membrane may be made of, for example, one or two or more layers. When forming the intervening membrane with a gold surface, the intervening membrane is preferably made of two layers, for example, and the gold membrane is preferably formed on the MSS membrane via a metal membrane for adhesion (adhesive membrane) from the viewpoint of improving the adhesiveness of the gold membrane. Examples of the metal of the adhesive membrane include titanium and chromium. The thickness of the adhesive membrane is, for example, 0.1 to 10 nm, and the thickness of the gold membrane is, for example, 0.1 to 100 nm. When the biotin is bonded to the intervening membrane, for example, a thiol alkane to which the biotin is bonded may be further used to form a self-assembled monolayer (SAM) of thiol alkane on the surface of the intervening membrane, and the aptamer to which the biotin is bonded may be contacted, and the aptamer may be immobilized by binding the biotin with the avidin.

As a third example, there is a method of binding the streptavidin to the MSS membrane by binding an amino group to the MSS membrane and further binding glutaraldehyde thereto. That is, a silane coupling agent having an amino group is reacted with the MSS membrane to bind an amino group to the MSS membrane. The reaction can be carried out, for example, by applying a solution containing a silane coupling agent having an amino group to the MSS membrane. Further, a crosslinking agent capable of binding an amino group and a main chain or a side chain of an amino acid is reacted with the MSS membrane, or a crosslinking agent such as glutaraldehyde capable of forming a linker between an amino group and a main chain or a side chain of an amino acid is reacted with the MSS membrane, thereby binding one end of a crosslinking agent such as glutaraldehyde to the amino group on the MSS membrane. Specifically, the membrane surface of the MSS membrane after silane coupling is washed, a solution containing a crosslinking agent is applied to the MSS membrane to bind the amino group to the crosslinking agent. The conditions of the crosslinking reaction can be appropriately determined, for example, depending on the type of the crosslinking agent. Next, the avidin is bonded to the other end of the crosslinking agent such as glutaraldehyde. Specifically, the membrane surface of the MSS membrane after crosslinking is washed, and a solution containing the avidin is applied to bind the other end of the crosslinking agent to the main chain or the side chain of the amino acid of the avidin. Then, an aptamer to which the biotin is bonded is brought into contact with the MSS membrane thus treated, and the aptamer can be immobilized by binding the biotin with the avidin.

A silane coupling agent is represented by Y—Si (CH₃)₃₋ₙ(OR)ₙ, for example. When the silane coupling agent is a silane coupling agent having an amino group, n, R, and Y may be as described below, for example The “n” is 2 or 3. Examples of the “R” include alkyl groups such as a methyl group, an ethyl group, and the like; and acyl groups such as an acetyl group, a propyl group, and the like. The “Y” is a reactive functional group having an amino group at its end.

Examples of the silane coupling agent having the amino group include N-(2-aminoethyl)-3-aminopropylmethyldimethoxysilane (for example, KBM-602 (produced by Shin-Etsu Silicone Co., Ltd.)), N-(2-aminoethyl)-3-aminopropyltrimethoxysilane (for example, KBM-603 (produced by Shin-Etsu Silicone Co., Ltd.)), 3-aminopropyltriethoxysilane (for example, KBM-903 (produced by Shin-Etsu Silicone Co., Ltd.)), 3-aminopropyltriethoxysilane (for example, KBE-903 (produced by Shin-Etsu Silicone Co., Ltd.)), 3-(2-aminoethylamino)propyltrimethoxysilane (for example, GENIOSIL® GF 91 (produced by Wacker Asahikasei Silicone Co., Ltd.), and 3-(2-aminoethylamino)propylmethyldimethoxysilane (for example, GENIOSIL® GF 95 (produced by Wacker Asahikasei Silicone Co., Ltd.).

The crosslinking agent can be appropriately determined depending on the functional group of the main chain or the side chain of the amino acid to be bonded to the linker. Examples of the functional group include an amino group (—NH₂), a thiol group (—SH), and a carboxyl group (—COOH). The amino group has, for example, an N-terminal of protein or peptide or a side chain of lysine. The thiol group has, for example, a side chain of cysteine. The carboxyl group has, for example, a C-terminal of protein or peptide or a side chain of aspartic acid or glutamic acid.

When an amino group of the main chain or the side chain of the amino acid is utilized, examples of the crosslinking agent include a crosslinking agent having aldehyde groups such as glutaraldehyde and the like at both ends; a crosslinking agent having N-hydroxysuccinimide active esters (N-hydroxysuccinimide reactive groups) such as bis(sulfosuccinimidyl)suberate (BS3), disuscinimidyl glutarate (DSG), disuscinimidyl suberate (DSS), dithiobis(succinimidyl propionate), dithiobis(sulfosuccinimidyl propionate) (DSP), dithiobis(succinimidyl propionate) (DTSP), dithiobis(sulfosuccinimidyl propionate) (DTSSP), dissuccinimidyl tartrate (DST), ethylene glycol bis(succinimidyl succinate) (ESG)), ethylene glycol bis(sulfosuccinimidyl succinate) (Sulfo-ESG), PEGylated bis(sulfosuccinimidyl) (BS(PEG)5, BS(PEG)9, etc.), and the like at both ends; and a crosslinking agent having imide ester reactive groups such as dimethyl adipoimidate (DMA), dimethyl pimelimidate (DMP), dimethyl pimelimidate (DMS), and the like at both ends.

When a thiol group of the side chain of the amino acid is utilized, examples of the crosslinking agent include a crosslinking agent having a N-hydroxysuccinimide active ester and a maleimide group such as N-(6-maleimide caproyloxy) succinimide (EMCS), N-(6-maleimide caproyloxy) sulfosuccinimide (Sulfo-EMCS), N-(8-maleimide capryloxy) succinimide (HMCS), N-(8-maleimide sulfocapryloxy) (Sulflo-HMCS), N-α-maleimidoacet-oxysuccinimide ester (AMAS), N-β-maleimidopropyl-oxysuccinimide ester (BMPS), N-γ-maleimidobutyryl-oxysuccinimide ester (GMBS), N-γ-maleimidobutyryl-oxysulfosuccinimide ester (Sulfo-GMBS), m-maleimidobenzoyl-N-hydroxysuccinimide ester (MBS), m-maleimidobenzoyl-N-hydroxysulfosuccinimide ester (Sulfo-MBS), succinimidyl 4-(N-maleimidomethyl)cyclohexane-1-carboxylate (SMCC), sulfosuccinimidyl 4-(N-maleimidomethyl)cyclohexane-1-carboxylate (Sulfo-SMCC), succinimidyl 4-(p-maleimidophenyl)butyrate (SMPB), sulfosuccinimidyl 4-(N-maleimidophenyl)butyrate (Sulfo-SMPB), Succinimidyl 6-((beta-maleimidopropionamido) hexanoate) (SMPH), succinimidyl 4-(N-maleimidomethyl)cyclohexane-1-carboxy-(6-amidocaproate) (LC-SMCC), N-K-maleimidoundecanoyl-oxysulfosuccinimide ester (Sulfo-KMUS), or the like at both ends of the molecule; a crosslinking agent having a N-hydroxysuccinimide active ester and a haloacetyl reactive group such as succinimidyl iodoacetate (SIA), succinimidyl 3-(bromoacetamido)propionate (SBAP), succinimidyl(4-iodoacetyl)aminobenzoate (SIAB), sulfosuccinimidyl(4-iodoacetyl)aminobenzoate (Sulfo-SIAB), or the like; and a crosslinking agent having pyridyl dithiol reactive group and a N-hydroxysuccinimide active ester such as succinimidyl 3-(2-pyridyldithio)propionate (SPDP), succinimidyl 6-(3(2-pyridyldithio)propionamido)hexanoate (LC-SPDP), sulfosuccinimidyl 6-(3 (2-pyridyldithio)propionamido)hexanoate (Sulfo-LC-SPDP), 4-succinimidyloxycarbonyl-alpha-methyl-α(2-pyridyldithio)toluene (SMPT), 2-pyridyldithiol-tetraoxatetradecane-N-hydoxysuccinimide (PEG4-SPDP), 2-pyridyldithiol-tetraoxaoctatriacontane-N-hydoxysuccinimide (PEG12-SPDP)), or the like at both ends.

When a carboxyl group of the main chain or the side chain of the amino acid is utilized, examples of the crosslinking agent include dicyclohexylcarbodiimide (DCC), 1-ethyl-3-(3-dimethylaminopropyl)carbodiimide (EDC), N-hydroxysuccinimide (NHS), N-hydroxysulfosuccinimide (Sulfo-NHS), and acetic anhydride. Note that since the amino group and the carboxyl group are directly bonded, DCC, EDC, NHS, Sulfo-NHS, and acetic anhydride do not remain between the carboxyl group and the amino group, and a linker region (group) derived from a crosslinking agent is not formed.

The crosslinking agent is preferably a crosslinking agent in which self-condensation does not substantially occur because the length of the linker can be kept at a substantially constant length or a constant length. “The length of the linker is constant” means that, for example, in a linker of a plurality of aptamers, the length of the linker of each of the aptamers is substantially the same or the same. The length of the linker can be made substantially the same or the same, for example, by making the structure of the linker substantially the same or the same. In the third example, by using such a crosslinking agent, the sensitivity of the MSS can be improved. The improvement of the sensitivity is presumed to be due to the following reasons. Note that the present invention is not limited in any way to the following presumption. By binding of the target to the aptamer, steric hindrance due to the target occurs around the aptamer to which the target is bonded. If the aptamers are immobilized at different distances to the MSS membrane, the target is likely to contact an aptamer present on the distal end side from the MSS membrane. Thus, it is presumed that the target preferentially binds to an aptamer on the distal end side from the MSS membrane. In this case, even if steric hindrance due to the target occurs around the aptamer to which the target is bonded, the other aptamers are less susceptible to steric hindrance due to the target because many of them are present on the MSS membrane side as compared to the aptamer to which the target is bonded. Therefore, even when the target binds to an aptamer, there is a low possibility that the positions of the surrounding aptamers move due to steric hindrance. Thus, when the aptamers are immobilized at different distances to the MSS membrane, there is also a relatively low possibility of distortion of the MSS membrane due to movement of the positions of the surrounding aptamers. That is, aptamers surrounding the aptamer to which the target is bonded are less likely to move due to the binding and the distortion of the MSS membrane is less likely to be increased. On the other hand, when the aptamers are immobilized at substantially the same distance to the MSS membrane, upon binding of the target to the aptamer, the aptamers surrounding the aptamer to which the target is bonded are affected by steric hindrance due to the target. Therefore, the positions of the surrounding aptamers are likely to move, and the distortion of the MSS membrane is relatively likely to occur due to the movement of the positions of the surrounding aptamers. That is, when the aptamers are immobilized at substantially the same distance to the MSS membrane, the binding between one aptamer and the target causes the movement of the positions of the surrounding aptamers on the MSS membrane, thereby increasing the distortion of the MSS membrane. Therefore, when the aptamers are immobilized at substantially the same distance to the MSS membrane, that is, when the length of the linker is kept at a substantially constant length, it is presumed that the sensitivity of the MSS membrane improves.

Specific examples of the crosslinking agent in which the self-condensation does not substantially occur include a crosslinking agent having N-hydroxysuccinimide active esters at both ends, a crosslinking agent having imide ester reaction groups at both ends, a crosslinking agent having a maleimide group and a N-hydroxysuccinimide active ester at both ends of the molecule, a crosslinking agent having a N-hydroxysuccinimide active ester and a haloacetyl reactive group at both ends, a crosslinking agent having a N-hydroxysuccinimide active ester and a pyridyldithiol reactive group at both ends, DCC, EDC, NHS, Sulfo-NHS, and acetic anhydride.

The linker is represented by, for example, the following formula (1). In the following formula (1), M₁ represents an atom bonded to the silane coupling agent on the MSS membrane, L₁ represents a region (group) derived from a silane coupling agent, L₂ represents a region (group) derived from the cross-linking agent, L₂ is optional, and M₂ represents an atom bonded to a cross-linking agent or NH in the affinity tag. Further, NH represents an amine derived from an amino group of a silane coupling agent having an amino group.

M₁-L₁-NH-L₂-M₂ (1)

L₁ is represented by (M₁)-Si(CH₃)₂₋ₘ(OR₄)ₘ—R₁—(NH) or (M₁)-Si(CH₃)₂₋ₘ(OR₄)ₘ—R₂—NH—R₃—(NH), for example. R₁ is a straight or branched alkyl group having 1 to 5 carbon atoms. R₂ and R₃ are, for example, each independently a straight or branched alkyl group having 1 to 5 carbon atoms and may be the same or different from each other. Examples of the alkyl group include a methyl group, an ethyl group, a propyl group, a butyl group, and a pentyl group. R₄ is, for example, a hydrogen atom or a bond, and m is 1 or 2.

When 3-aminopropyltriethoxysilane is used as the silane coupling agent, L₁ is represented by, for example, (M₁)-Si(OR₄)₂—(CH₂)₃—(NH). R₄ is, for example, a hydrogen atom or a bond. Further, when glutaraldehyde is used as the crosslinking agent, L₂ is represented by, for example, (NH)═CH—C₃H₆—CH═(CH(CHO)—C₂H₄—CH)ₙ═CH(CHO)—C₂H₄—C═(M₂).

The length of the linker may be expressed, for example, by a length of a shortest molecular chain (main chain length) from a functional group on a MSS membrane (e.g., an oxygen atom of a silanol group on a silicon membrane) to an affinity tag such as avidin or an aptamer. The main chain length of the linker is 1 to 20 and is preferably 1 to 15, 1 to 13, 3 to 13, 5 to 13, 1 to 11, 3 to 11, 1 to 10, 3 to 10, 1 to 8, 3 to 8, 1 to 5, 1 to 3, 1 or 2 because the sensitivity of the MSS can be improved.

Note that, while the avidin-biotin binding is utilized in the third example, the third example is not limited thereto, and a linker may be directly bonded to a hydroxyl group or a phosphate group of the aptamer. In this case, the aptamer can be immobilized to the MSS membrane by amididizing a phosphate group at the 3′ end to react with the linker.

A fourth example is a method of binding a methacrylic group (—C(═O)—C(CH₃)═CH₂) to the MSS membrane, and binding the streptavidin to the MSS membrane via an amino acid or a derivative of the methacrylic group (hereinafter, referred to as an “amino acid derivative”). That is, a silane coupling agent having a methacrylic group is reacted with the MSS membrane to bind an amino group to the MSS membrane. The reaction can be carried out, for example, by applying a solution containing a silane coupling agent having a methacrylic group to the MSS membrane. Further, an amino acid derivative such as N-acetylcysteine is reacted with the MSS membrane, and then a crosslinking agent capable of forming a linker between a main chain or a side chain of the amino acid derivative and a main chain or a side chain of the amino acid of the avidin is reacted with the MSS membrane thereby binding one end of the crosslinking agent to the amino acid derivative on the MSS membrane. Specifically, the membrane surface of the MSS membrane after treatment with the amino acid derivative is washed, a solution containing a crosslinking agent is applied to the MSS membrane to bind the amino acid derivative to the crosslinking agent. The conditions of the crosslinking reaction can be appropriately determined, for example, depending on the type of the crosslinking agent. Next, the avidin is bonded to the other end of the crosslinking agent. Specifically, the membrane surface of the MSS membrane after crosslinking is washed, and a solution containing avidin is applied to bind the other end of the crosslinking agent to the main chain or the side chain of the amino acid of the avidin. Then, an aptamer to which the biotin is bonded is brought into contact with the MSS membrane thus treated, and the aptamer can be immobilized by binding the biotin with the avidin. Note that, while the avidin-biotin binding is utilized in the fourth example, the fourth example is not limited thereto, and a linker may be directly bonded to a hydroxyl group or a phosphate group of the aptamer.

As described above, the silane coupling agent is represented by Y—Si (CH₃)₃₋ₙ(OR)ₙ, for example. When the silane coupling agent is a silane coupling agent having a methacrylic group, n, R, and Y may be as described below, for example. The “n” is 2 or 3. Examples of the “R” include alkyl groups such as a methyl group, an ethyl group, and the like; and acyl groups such as an acetyl group, a propyl group, and the like. The “Y” is a reactive functional group having a methacrylic group at its end.

Examples of the silane coupling agent having a methacrylic group include 3-(methacryloyloxy)propylmethyldimethoxysilane (e.g., KBM-502 (produced by Shin-Etsu Silicone Co., Ltd.)), 3-(methacryloyloxy)propyltrimethoxysilane (e.g., KBM-503 (produced by Shin-Etsu Silicone Co., Ltd.), GENIOSIL® GF3I (produced by Wacker Asahikasei Silicone Co., Ltd..)), 3-(methacryloyloxy)propylmethyldimethoxysilane (e.g., KBE-502 (produced by Shin-Etsu Silicone Co., Ltd.)), and (3-methacryloyloxypropyl)triethoxysilane (e.g., KBE-503 (produced by Shin-Etsu Silicone Co., Ltd.)).

The amino acid or amino acid derivative has, for example, a functional group capable of reacting with a methacrylic group and a carboxyl group. The functional group capable of reacting with the methacrylic group may be, for example, a thiol group (—SH). Examples of the amino acid or amino acid derivative having the thiol group include cysteine; and cysteine having a modified amino group such as N-acetylcysteine.

The crosslinking agent can be appropriately determined depending on, for example, a functional group of the amino acid derivative to be subjected to crosslinking and a functional group of an amino acid of the avidin to be subjected to crosslinking. As a specific example, when two functional groups are amino groups, regarding the crosslinking agent, reference can be made to the description as to a crosslinking agent when utilizing an amino group of a main chain or a side chain of the amino acid in the third example. Also, when one of two functional groups is an amino group and the other is a thiol group, regarding the crosslinking agent, reference can be made to the description as to a crosslinking agent when utilizing a thiol group of a side chain of the amino acid in the third example. Further, when one of two functional groups is an amino group and the other is a carboxyl group, regarding the crosslinking agent, reference can be made to the description as to a crosslinking agent when utilizing a carboxyl group of a main chain or a side chain of the amino acid in the third example.

The crosslinking agent is preferably a crosslinking agent in which self-condensation does not substantially occur because the length of the linker can be kept at a constant length. In the fourth example, by using such a crosslinking agent, the sensitivity of the MSS can be improved by the same mechanism as that described in the third example described above. Specific examples of the crosslinking agent in which self-condensation does not substantially occur include a crosslinking agent having N-hydroxysuccinimide active esters at both ends, a crosslinking agent having imide ester reaction groups at both ends, a crosslinking agent having a maleimide group and a N-hydroxysuccinimide active ester at both ends of the molecule, a crosslinking agent having a N-hydroxysuccinimide active ester and a haloacetyl reactive group at both ends, a crosslinking agent having a N-hydroxysuccinimide active ester and a pyridyldithiol reactive group at both ends, DCC, EDC, NHS, Sulfo-NHS, and acetic anhydride.

The linker is represented by, for example, the following formula (2). In the following formula (2), M₁ represents an atom bonded to the silane coupling agent on the MSS membrane, L₁ represents a region (group) derived from the silane coupling agent, A represents an amino acid derivative, L₂ represents a region (group) derived from the cross-linking agent, L₂ is optional, and M₂ represents an atom bonded to the cross-linking agent or NH in the affinity tag.

M₁-L₁-A-L₂-M₂ (2)

L₁ is represented by, for example, (M₁)-Si(CH₃)₂₋ₘ(OR₄)ₘ—R₅—C(═O)—CH₁(CH₃)₂₋ₗ-(A). R₄ is, for example, a hydrogen atom or a bond. R₅ is a straight or branched alkyl group having 1 to 5 carbon atoms. Examples of the alkyl group include a methyl group, an ethyl group, a propyl group, a butyl group, and a pentyl group. m is 1 or 2. l is 0 or 1.

When 3-(methacryloyloxy)propyltrimethoxysilane is used as the silane coupling agent, L₁ is represented by, for example, (M₁)-Si(OR₄)₂—(CH₂)₃—O—C(═O)—C(CH₃)₂-(A). R₄ is, for example, a hydrogen atom or a bond. In addition, when N-acetylcysteine is used as the amino acid derivative, A is represented by, for example, (L₁)-S—CH₂—CH(NH—COCH₃)—C(═O)-(M₂). When acetic anhydride is used as the crosslinking agent, L₂ is not present, for example.

The length of the linker may be expressed, for example, by a length of a shortest molecular chain (main chain length) from a functional group on a MSS membrane (e.g., a silanol group on a silicon membrane) to an affinity tag such as avidin. The main chain length of the linker is 1 to 20 and is preferably 1 to 15, 1 to 13, 1 to 11, 1 to 10, 1 to 8, 1 to 5, 1 to 3, 1 or 2 because the sensitivity of the MSS can be improved.

Note that, while the avidin-biotin binding is utilized in the fourth example, the fourth example is not limited thereto, and a linker may be directly bonded to a hydroxyl group or a phosphate group of the aptamer. In this case, the aptamer can be immobilized to the MSS membrane by amididizing a phosphate group at the 3′ end to react with the linker.

The immobilization site of the aptamer to the MSS membrane is not particularly limited, and may be, for example, the 3′ end or the 5′ end.

In the MSS of the present example embodiment, the sensor substrate has a support region for supporting the MSS membrane, and the support region has a piezoresistive element. The sensor substrate supports the MSS membrane by the support region. In the MSS membrane, for example, the aptamer is immobilized to one or both surfaces facing each other as described above, and supported at the side by the sensor substrate. It is preferable that the sensor substrate partially support the MSS membrane, for example, and in particular, it is preferable that the sensor substrate partially support the side surface of the MSS membrane. In the MSS membrane, the number of positions (supporting portions) supported by the supporting region of the sensor substrate is not particularly limited, and is, for example, four. This description however is a mere example and does not limit the present invention at all.

In the sensor substrate, the support region is, for example, a silicon membrane, and by making any region of the silicon membrane into p-type by doping an impurity, the p-type region (p-type Si) can be functioned as the piezoresistive element. The support region has the piezoresistive element at or in the vicinity of the position supporting the MSS membrane, for example. The sensor substrate may be made entirely of silicon, or only the support region may be a silicon membrane, for example, and materials of the sensor substrate other than the support region having the piezoresistive element are not particularly limited.

In the MSS of the present example embodiment, for example, the sensor substrate has a circuit for applying a voltage. When the support region supports the MSS membrane at a plurality of points and has piezoresistive elements at or in the vicinity of the positions supporting the MSS membrane, for example, the circuit may be, for example, a Wheatstone bridge circuit having a plurality of piezoresistive elements in the support region. As to the MSS of the present example embodiment, for example, by applying a voltage to the Wheatstone bridge circuit, as described above, it is possible to measure the electrical signal accompanying the change in resistance value in the piezoresistive element.

In the MSS of the present example embodiment, for example, the sensor substrate may have a plurality of the support regions, and the plurality of the support regions may each support the MSS membrane. In the MSS of the present example embodiment, the number of the support regions and the number of the MSS membranes to be supported are not particularly limited, and may be one or two or more. When the MSS of the present example embodiment has a plurality of the MSS membranes, the plurality of MSS membranes may be, for example, MSS membranes having aptamers for the same target immobilized thereon, MSS membranes having aptamers for different targets immobilized thereon, or MSS membranes having both the aptamers for the same target and the aptamers for different targets immobilized thereon. Here, the “aptamers for the same target” may be, for example, aptamers having the same sequence for the same target, or aptamers having different sequences for the same target.

When the MSS of the present example embodiment has a plurality of MSS membranes having aptamers for the same target immobilized thereon, for example, a plurality of analyses for the same target can be performed at the same time with one MSS. In addition, when the MSS of the present example embodiment has a plurality of MSS membranes having aptamers for different targets immobilized thereon, for example, analyses for different targets can be performed at the same time with one MSS.

The MSS of the present example embodiment may be in a form in which the MSS membrane is placed on the sensor substrate at the time of use, for example, and the sensor substrate and the MSS membrane may be separately independent before use. In the latter case, for example, the MSS of the present invention may be, for example, a kit including the sensor substrate and the MSS membrane separately and independently.

The MSS of the present example embodiment can measure a change in resistance value due to a stress change of the piezoresistive element in the MSS as an electronic signal by using an existing measurement module in an analysis method described below, for example.

### Second Example Embodiment

As described above, a method for analyzing a target of the present example embodiment is characterized in that it includes the steps of: immersing the membrane-type surface-stress sensor (MSS) according to the present invention in a sample liquid; applying a voltage to the MSS in a liquid phase; and analyzing a target in the sample liquid by measuring a stress change of the piezoresistive element in the MSS. The analysis method of the present invention is characterized in that the MSS having aptamers immobilized thereon is used as described above, and other steps, conditions, and the like are not particularly limited.

In the immersing, the support region having the piezoresistive element of the sensor substrate and the MSS membrane supported by the support region of the MSS may be immersed in the sample liquid, for example. The conditions for immersing the MSS in the sample liquid are not particularly limited, and the immersion may be performed, for example, 0.1 to 120 minutes at a temperature from 20 to 35° C. and 0.1 to 120 minutes at a temperature from 50 to 60° C. When the MSS has a plurality of MSS membranes, for example, a plurality of MSS membranes in the MSS may be simultaneously immersed in the same sample liquid.

In the voltage-applying, as described above, a voltage is applied to the membrane-type surface-stress sensor in the liquid phase. The application conditions of the voltage are not particularly limited, and for example, the same conditions as those of a commercially available MSS can be exemplified. The liquid phase may be, for example, a sample liquid in the immersing, or other solvents. In the latter case, the MSS after the immersing may be taken out from the sample liquid and immersed in a new solvent, and then a voltage may be applied. When the MSS is washed for removing a substance that has not bonded to the aptamer in the sample after the immersing in the sample liquid, it is preferable to immerse the MSS in such a new solvent and to apply a voltage as described above. The solvent is not particularly limited, and examples thereof include buffers such as PBS, Tris-HCl, and the like; and water.

In the analyzing, as described above, the target in the sample liquid is analyzed by measuring the stress change of the piezoresistive element in the MSS. The measurement of the stress change can be performed, for example, by measuring an electric signal using a commercially available measurement module (for example, MSS-8RM, NANOSENSORS).

### EXAMPLES

### Example 1

Aptamers were immobilized to a commercially available MSS membrane to examine whether the analysis of the target was possible.

(1) Immobilization of Aptamers by Nonspecific Adsorption

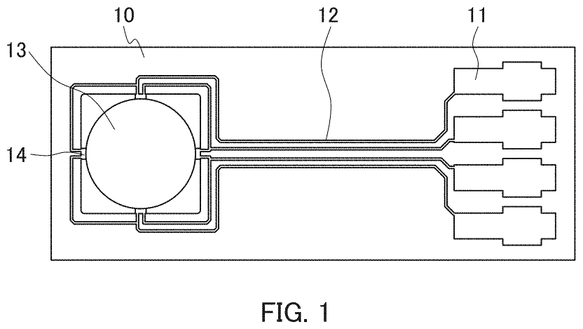

A commercially available MSS (trade name: SD-MSS-1K2G, NANOSENSORS) was used. The configuration of the MSS is shown in the top view of FIG. 1. In the MSS, as shown in FIG. 1, the sensor substrate 10 includes an electrode 11, an aluminum wire 12, a MSS membrane 13, and a piezoresistive element 14, the MSS membrane 13 is connected to the aluminum wire 12 via the piezoresistive element 14, and the aluminum wire 12 is connected to the electrode 11.

An acrylic resin (trade name: Mr. COLOR 62, produced by GSI Creos Corporation) and an epoxy resin (trade name: PM 165-R Hi, produced by CEMEDINE CO., LTD.) were applied to the aluminum wire on the sensor substrate to perform a waterproofing treatment. The sensor substrate after the treatment was connected to a substrate with a connector (trade name: IFB-FFC (0.5) 4P-B, produced by AITENDO) so that the electrode was inserted into the sensor substrate. Further, the waterproofing treatment was applied to the entire connector of the substrate with the connector by applying the same resin as described above to the exposed portion of the metal, the gap connected to the metal portion, and the like to fill up with the resin.

Next, 1 μl of a streptavidin solution was added dropwise to the back surface (the surface opposite to the surface on which the aluminum wire was formed) of the MSS membrane of the sensor substrate, and the sensor substrate was allowed to stand for 1 hour at room temperature (about 25° C.) under a water vapor atmosphere (100% (relative humidity)). The streptavidin solution was prepared by suspending in 1×PBS (pH 7.4) so as to achieve a streptavidin concentration of 2%. Next, after washing the back surface with the PBS, 3 μl of the streptavidin solution was added dropwise to the surface of the MSS membrane, allowed to stand under the same conditions, and further washed with the PBS. Then, the sensor substrate was dried at room temperature for 10 minutes.

Two of the sensor substrates were treated in this manner, and aptamers were further bonded to one of the sensor substrates to provide the MSS of Example 1A, and poly-T was further added to the other of the sensor substrates to provide the MSS of Reference Example 1A.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending thrombin aptamer having a biotin tag added to the 3′ end (SEQ ID NO: 1: GGTTGGTGTGGTTGGTTTTT-biotin-3′) in the PBS so as to have a final concentration of 1 μmol/l. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Example 1A.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of poly T having a biotin tag added to the 3′ end (SEQ ID NO: 2: TTTTTTTTTTTTTTTTTTTT-biotin-3′) in the PBS so as to have a final concentration of 1 μmol/l. Since the poly T has a biotin tag, if streptavidin is bonded to the surface of the MSS membrane, the poly T is immobilized to the surface of the MSS membrane by the binding between the biotin and the streptavidin. This was referred to as the MSS of Reference Example 1.

(2) Immobilization of Aptamers by Gold Deposition

The same treatment as in (1) above was performed, unless otherwise indicated. That is, the waterproofing treatment was applied to the aluminum wire on the sensor substrate of the commercially available MSS, then the electrode of the sensor substrate was masked, titanium deposition was performed on the entire surface of the sensor substrate including the MSS membrane, and then gold deposition was further performed. A titanium thin membrane having a thickness of about 5 nm was formed by the titanium deposition, and a gold thin membrane having a thickness of about 100 nm was formed by the gold deposition. The sensor substrate was then washed with ethanol.

Next, the entire sensor substrate was immersed in 100 μmol/l BiotinSAM ethanol solution (Dojindo Laboratories), allowed to stand at room temperature for 1 hour, and further washed with ethanol. Then, in the same manner as in (1) above, the sensor substrate was connected to the substrate with a connector, and the entire connector was subjected to the waterproofing treatment.

Next, 1 μl of a 0.5% streptavidin solution was added dropwise to one surface (the surface on which the aluminum wire was formed) of the MSS membrane of the sensor substrate, and the sensor substrate was allowed to stand for 0.5 hours at room temperature under a water vapor atmosphere (100% (relative humidity)). Next, the sensor substrate was washed with the PBS, and then was dried at room temperature for 10 minutes.

Two of the sensor substrates were treated in this manner, and aptamers were further bonded to one of the sensor substrates to provide the MSS of Example, and poly-T was further added to the other of the sensor substrates to provide the MSS of Reference Example.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmol/l. After standing, the sensor substrate was further immersed in PBS containing 1% BSA, allowed to stand at room temperature for 50 minutes, and washed with the PBS. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Example 2.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 50 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of Reference Example 2.

(3) Immobilization of Aptamers by Silane Coupling

The sensor substrate of the commercial MSS was washed with ethanol, the end of the sensor substrate on which the MSS membrane was placed was immersed in a silane coupling solution and allowed to stand for 20 minutes at room temperature. The composition of the silane coupling agent was as follows: 8 ml of ethanol, 200 μl of acetic acid, 10 μl of APTMS (3-aminopropyltriethoxysilane), and 1.8 ml of pure water. Then, the immersed end of the sensor substrate was washed with pure water, and the treatment was performed at 110° C. for 1.5 hours.

In the same manner as in the (1), the waterproofing treatment was applied to the aluminum wire on the sensor substrate, then the sensor substrate was connected to the substrate with a connector, and the entire connector was subjected to the waterproofing treatment.

Next, 1 μl of a 14% glutaraldehyde solution was added dropwise to one surface (the surface on which the aluminum wire was formed) of the MSS membrane of the sensor substrate, and the sensor substrate was allowed to stand for 0.75 hours at room temperature under a water vapor atmosphere (100% (relative humidity)). Next, the sensor substrate was washed with the PBS, and then was dried at room temperature for 10 minutes. Further, 1 μl of a 0.5% streptavidin solution was added dropwise to the same surface of the MSS membrane of the sensor substrate, and the sensor substrate was allowed to stand for 0.5 hours at room temperature under a water vapor atmosphere (100% (relative humidity)). Next, the surface of the end of the sensor substrate on which the MSS membrane was placed was washed with 0.1 mol/l Tris-HCl (pH8), and then further immersed in 0.1 mol/l Tris-HCl (pH8) and allowed to stand at room temperature for 15 minutes. Thereafter, the end of the sensor substrate was washed with the PBS.

Two of the sensor substrates were treated in this manner, and aptamers were further bonded to one of the sensor substrates to provide the MSS of Example, and poly-T was further added to the other of the sensor substrates to provide the MSS of Reference Example.

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmol/l. After standing, the sensor substrate was further immersed in PBS containing 1% BSA, allowed to stand at room temperature for 30 minutes, and washed with the PBS. Since the thrombin aptamer has a biotin tag, if streptavidin is bonded to the surface of the MSS membrane, the thrombin aptamer is immobilized to the surface of the MSS membrane by the binding between the biotin and the streptavidin. This was referred to as the MSS of Example 3.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 30 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of Reference Example 3.

(4) Detection of Electrical Signal

Each set of the MSSs of Example 1 and Reference Example 1, the MSSs of Example 2 and Reference Example 2, and the MSSs of Example 3 and Reference Example 3 was immersed in a sample liquid at the same time, and a voltage was applied to measure a voltage change accompanying a stress change. Specifically, first, the end of the MSS including the MSS membrane was immersed in the PBS, a voltage was applied to the MSS, and the MSS was left to stand until the signal of the voltage was stabilized. Then, at a measurement time of 1400 seconds when the signal of the voltage was sufficiently stabilized, the solution of the immersion of the MSS was switched from the sample liquid to the thrombin solution, and the signal of the voltage was continuously measured. The thrombin solution was prepared by mixing a thrombin reagent (trade name: αThrombin, Human, Funakoshi Co., Ltd..) in the PBS so as to have a final concentration of 240 nmol/l.

Then, the voltage signal was converted into a voltage for each of the MSSs, and the difference (Vs−Vt) between the stable voltage (Vs) after immersion in the sample liquid and the lowest voltage (Vt) after immersion in the thrombin solution was obtained, and the difference was determined to be the drop voltage (N) due to immersion in the thrombin solution. These results are summarized in Table 1 below.

TABLE 1

| | | Drop voltage | |
|---|---|---|---|
| | | value (electrical | Drop voltage |
| | | signal) | value (μV) |
| Nonspecific | Example 1 | 142 | 469 |
| adsorption | Reference Example 1 | 76 | 251 |
| Gold | Example 2 | 149 | 492 |
| deposition | Reference Example 2 | 123 | 406 |
| Silane | Example 3 | 77 | 254 |
| coupling | Reference Example 3 | 36 | 119 |

A sharp decrease in voltage was observed after immersion in a thrombin solution in all of the MSSs of Example 1, Example 2, and Example 3. The drop voltage of each Example showed a significantly larger value (large drop in voltage) than the drop voltage of each corresponding Reference Example, as summarized in Table 1. This result shows that, in the MSSs of Examples, thrombin was bonded to the MSS membrane of the MSS via an aptamer due to the immersion in a target thrombin solution, which caused a stress change. Note that, while a decrease in the measurement value of the electric signal was also observed after switching to the thrombin solution in each Reference Example as summarized in Table 1, this is an influence of glycerin contained in the thrombin reagent used (an influence such as non-specific adsorption or the like of glycerin onto the MSS membrane), which can be neglected by background correction. Further, since each Example showed a voltage drop larger than that of each corresponding Reference Example, it is obvious that specific binding with thrombin as a target occurs in Examples, unlike Reference Examples. From the above results, it was verified that a target can be analyzed using the MSS having aptamers immobilized thereon.

### Example 2

The present example examined that the sensitivity of the MSS was improved by immobilizing aptamers at substantially the same distance to the MSS membrane.

(1) Production of MSS

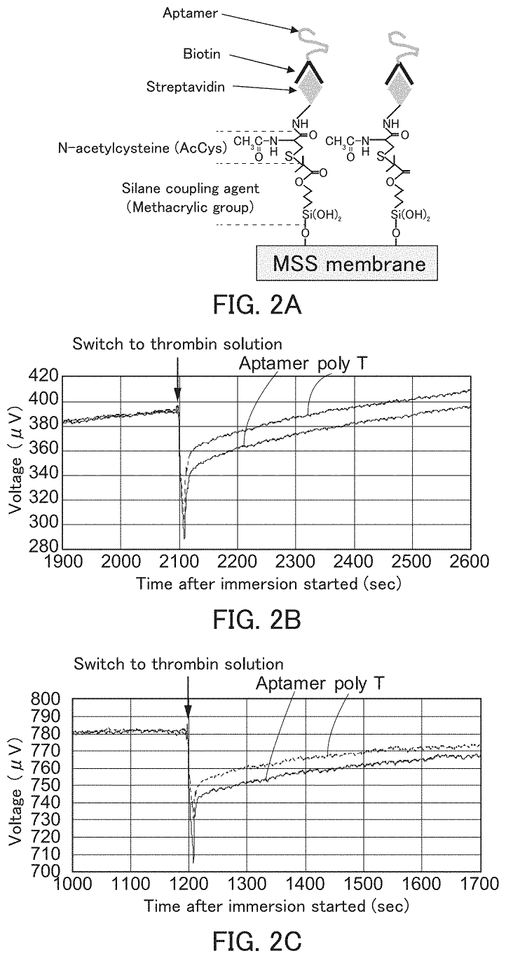

As a MSS of the present example, the MSS shown in (A) in FIG. 2 was produced. First, the sensor substrate of the commercially available MSS of Example 1(1) was washed with ethanol, then the end of the sensor substrate on which the MSS membrane was placed was rinsed with about 100 μl of a silane coupling solution and left at room temperature for 1.5 hours. The composition of the silane coupling agent was as follows: 8 ml of ethanol, 200 μl of acetic acid, 100 μl of APTMS (trimethoxylyl 3-propylmethacrylic acid(3-(methacryloyloxy)propyltrimethoxysilane), and 1.8 ml of pure water. Then, the sensor substrate was washed with ethanol and dried at room temperature for 5 minutes.

Next, 1 μl of a N-acetylcysteine solution was added dropwise to one surface of the MSS membrane of the sensor substrate (the surface on which the aluminum wire was formed), and the sensor substrate was irradiated with UV (Nitride Co., Ltd., NS365L-6SMG) for several minutes. The sensor substrate was irradiated with UV at room temperature under a water vapor atmosphere (100% (relative humidity)) until it was dried. Thereafter, the sensor substrate was washed with pure water and dried at room temperature.

Next, the sensor portion of the sensor substrate was immersed in an acetic anhydride solution (10% acetic anhydride, 90% acetonitrile) and reacted at 60° C. for 0.5 hours. After the reaction, the sensor substrate was washed with acetonitrile.

The sensor substrate was then dried at room temperature for 10 minutes. Then, 1 μl of a 0.5% streptavidin solution was added dropwise to the same surface of the MSS membrane of the sensor substrate, and the sensor substrate was allowed to stand for 1.5 hours at room temperature under a water vapor atmosphere (100% (relative humidity)). After standing, the sensor substrate was washed with the PBS.

In the same manner as in Example 1(1), the waterproofing treatment was applied to the aluminum wire on the sensor substrate, then the sensor substrate was connected to the substrate with a connector, and the entire connector was subjected to the waterproofing treatment.

Two of the sensor substrates were treated in this manner, and aptamers were further bonded to one of the sensor substrates to provide the MSS of Example (Example 2-1), and poly-T was further added to the other of the sensor substrates to provide the MSS of Reference Example (Reference Example 2-1).

In other words, 3 μl of an aptamer solution was added dropwise to the surface of the one of the sensor substrates and allowed to stand for 1 hour at room temperature under a water vapor atmosphere (100% (relative humidity)). The aptamer solution was prepared by suspending the thrombin aptamer in the PBS so as to have a final concentration of 5 μmol/l. After standing, the sensor substrate was further immersed in PBS containing 1% BSA, allowed to stand at room temperature for 30 minutes, and washed with the PBS. Since the thrombin aptamer has a biotin tag, if streptavidin is bonded to the surface of the MSS membrane, the thrombin aptamer is immobilized to the surface of the MSS membrane by the binding between the biotin and the streptavidin as shown in (A) in FIG. 2. This was referred to as the MSS of Example 2-1. The MSS of Example 2-1 corresponds to the MSS to which the aptamers are immobilized at substantially the same distance to the MSS membrane.

3 μl of a poly T solution was added dropwise to the surface of the other of the sensor substrates and allowed to stand for 1 hour at room temperature under a water vapor atmosphere (100% (relative humidity)). The poly T solution was prepared by suspending DNA of the poly T in the PBS so as to have a final concentration of 5 μmol/l. After standing, the sensor substrate was further immersed in PBS containing 1% BSA, allowed to stand at room temperature for 30 minutes, and washed with the PBS. Since the poly T has a biotin tag, if streptavidin is bonded to the surface of the MSS membrane, the poly T is immobilized to the surface of the MSS membrane by the binding between the biotin and the streptavidin. This was referred to as the MSS of Reference Example 2-1.

Further, in the same manner as in Example 1(1), the MSS of Example (Example 2-2) and the MSS of Reference Example (Reference Example 2-2) were produced.

(2) Detection of Electrical Signal

Each set of the MSSs of Example 2-1 and Reference Example 2-1 and the MSSs of Example 2-2 and Reference Example 2-2 was immersed in a sample liquid at the same time, and a voltage was applied to measure a voltage change accompanying a stress change. Specifically, first, the end of the MSS including the MSS membrane was immersed in the PBS, a voltage was applied to the MSS, and the MSS was left to stand until the signal of the voltage was stabilized. Then, at a measurement time of 1200 seconds or 2100 seconds when the signal of the voltage was sufficiently stabilized, the solution of the immersion of the MSS was switched from the sample liquid to the thrombin solution, and the signal of the voltage was continuously measured. The thrombin solution was prepared by mixing the thrombin reagent in the PBS so as to have a final concentration of about 200 nmol/l. The results are shown in FIG. 2.

FIG. 2 shows a schematic diagram showing the structure of the MSS and graphs showing the voltage of the MSS in Example 2. In FIG. 2, (A) is a diagram showing the structure of the MSS of Example 2-1, (B) is a graph showing the results of Example 2-1 and Reference Example 2-1, and (C) is a graph showing the results of Example 2-2 and Reference Example 2-2. In (B) and (C) in FIG. 2, the horizontal axis represents the time after the immersion of the end in PBS is started, and the vertical axis represents the voltage. As shown in (B) and (C) in FIG. 2, both MSSs of Example 2-1 and Example 2-2 showed a significantly higher voltage value than that of the MSSs of Reference Example 2-1 and Reference Example 2-2. This showed that the voltage changes depending on the presence or absence of the target. In other words, it was verified that a target can be detected using the MSS of the present invention. Further, the difference between the voltage value of the MSS of Example 2-1 and the voltage value of the MSS of Reference Example 2-1 was about twice as large as the difference between the voltage value of the MSS of Example 2-2 and the voltage value of the MSS of Reference Example 2-2. That is, the MSS of Example 2-1 exhibited twice the sensitivity of that of the MSS of Example 2-2.

Further, as summarized in Table 1, the MSS of Example 1 produced by the non-specific adsorption exhibits about twice the sensitivity of that of the MSS of Example 3 produced by the silane coupling. That is, the MSS of Example 2-1 exhibits about four times the sensitivity of Example 3 of Table 1. Further, while streptavidin is immobilized to a MSS membrane using a silane coupling agent in the MSS of Example 3 in Table 1, since glutaraldehyde is used as a crosslinking agent, aptamers are immobilized at different distances to the MSS membrane. Thus, it was presumed that the difference in sensitivity between the MSS of Example 2-1 and the MSS of Example 3 of Table 1 above was depending on whether or not the aptamers were immobilized at a certain distance to the MSS membrane. Further, since the main chain length of the linker in the MSS of Example 2-1 was 11, it was presumed that the sensitivity of the MSS can be improved by immobilizing the aptamers at a substantially constant distance to the MSS membrane and setting the linker length at that time around 11.

From the above results, it was verified that the sensitivity of a MSS was improved by immobilizing aptamers at substantially the same distance to a MSS membrane.

While the present invention has been described above with reference to illustrative example embodiments and examples, the present invention is by no means limited thereto. Various changes and variations that may become apparent to those skilled in the art may be made in the configuration and specifics of the present invention without departing from the scope of the present invention.

This application claims priority from Japanese Patent Application Nos. 2019-128311 filed on Jul. 10 2019 and 2020-056897 filed on Mar. 26, 2020. The entire subject matter of the Japanese Patent Applications is incorporated herein by reference.

(Supplementary Notes)

Some or all of the above example embodiments and examples may be described as in the following Supplementary Notes, but are not limited thereto.
(Supplementary Note 1)
A membrane-type surface-stress sensor, including:

aptamers;

a membrane; and

a sensor substrate, wherein

the aptamer is a nucleic acid molecule that binds to a target and is immobilized to the membrane,

the membrane is a membrane that deforms upon binding of the target to the aptamer,

the sensor substrate has a support region,

the support region supports the membrane and has a piezoresistive element, and

the piezoresistive element is an element for detecting deformation of the membrane.
(Supplementary Note 2)
The membrane-type surface-stress sensor according to Supplementary Note 1, wherein

the membrane is a silicon membrane.
(Supplementary Note 3)
The membrane-type surface-stress sensor according to Supplementary Note 1 or 2, wherein

the support region partially supports the membrane.
(Supplementary Note 4)
The membrane-type surface-stress sensor according to any one of Supplementary Notes 1 to 3, wherein

the aptamers are immobilized to one surface of the membrane.
(Supplementary Note 5)
The membrane-type surface-stress sensor according to any one of Supplementary Notes 1 to 3, wherein

the aptamers are immobilized to both surfaces of the membrane.
(Supplementary Note 6)
The membrane-type surface-stress sensor according to any one of Supplementary Notes 1 to 5, wherein

the aptamers are immobilized to the membrane via a conjugate of avidin or an avidin derivative and biotin or a biotin derivative.
(Supplementary Note 7)
The membrane-type surface-stress sensor according to any one of Supplementary Notes 1 to 6, including:

a metal membrane on the surface of the membrane, wherein

the aptamers are immobilized to the surface of the membrane via the metal membrane.
(Supplementary Note 8)
The membrane-type surface-stress sensor according to any one of Supplementary Notes 1 to 7, wherein

the aptamers are immobilized to the membrane surface via a linker.
(Supplementary Note 9)
The membrane-type surface-stress sensor according to Supplementary Note 8, wherein

a length of the linker in each of the aptamers is substantially constant.
(Supplementary Note 10)
The membrane-type surface-stress sensor according to Supplementary Note 8 or 9, wherein

the linker contains a silane coupling agent (a region derived from the silane coupling agent).
(Supplementary Note 11)
The membrane-type surface-stress sensor according to any one of Supplementary Notes 8 to 10, wherein

the linker contains a crosslinking agent (a region derived from the crosslinking agent).
(Supplementary Note 12)
The membrane-type surface-stress sensor according to any one of Supplementary Notes 8 to 11, wherein

a main chain length of the linker is 1 to 15.
(Supplementary Note 13)
The membrane-type surface-stress sensor according to any one of Supplementary Notes 1 to 12, wherein

the aptamers are immobilized to the surface of the membrane via a silane coupling agent (a region derived from the silane coupling agent).
(Supplementary Note 14)
The membrane-type surface-stress sensor according to any one of Supplementary Notes 1 to 13, wherein

the sensor substrate has a plurality of support regions, and

the plurality of support regions each supports the membrane.
(Supplementary Note 15)
The membrane-type surface-stress sensor according to Supplementary Note 14, wherein

the plurality of membrane-type surface-stress sensors include sensors having aptamers for different targets immobilized thereon.
(Supplementary Note 16)
The membrane-type surface-stress sensor according to any one of Supplementary Notes 1 to 15, wherein

the sensor substrate has a circuit,

the support region has a plurality of piezoresistive elements, and

the circuit is a Wheatstone bridge circuit having the plurality of piezoresistive elements.
(Supplementary Note 17)
A method for analyzing a target, including the steps of:

immersing the membrane-type surface-stress sensor according to any one of
Supplementary Notes 1 to 16 in a sample liquid;

applying a voltage to the membrane-type surface-stress sensor in a liquid phase; and

analyzing a target in the sample liquid by measuring a stress change of the piezoresistive element in the membrane-type surface-stress sensor.
(Supplementary Note 18)
The method according to Supplementary Note 17, wherein

in the voltage-applying, the liquid phase is the sample liquid, and

the voltage-applying is performed directly after the immersing.

### INDUSTRIAL APPLICABILITY

According to the present invention, as a new form of binding a target, by immobilizing aptamers to the membrane, the possibilities such as application to a target different from that of a conventional membrane-type surface-stress sensor and modification different from that of a conventional membrane-type surface-stress sensor can be expanded, for example.

### REFERENCE SIGNS LIST

10: sensor substrate
11: electrode
12: aluminum wire
13: MSS membrane
14: piezoresistive element

What is claimed is:

1. A membrane-type surface-stress sensor, comprising:
aptamers;
a membrane; and
a sensor substrate, wherein
the aptamer is a nucleic acid molecule that binds to a target and is immobilized to the membrane,
the membrane is a membrane that deforms upon binding of the target to the aptamer,
the sensor substrate has a support region,
the support region supports the membrane and has a piezoresistive element, and
the piezoresistive element is an element for detecting deformation of the membrane.

2. The membrane-type surface-stress sensor according to claim 1, wherein
the membrane is a silicon membrane.

3. The membrane-type surface-stress sensor according to claim 1, wherein
the support region partially supports the membrane.

4. The membrane-type surface-stress sensor according to claim 1, wherein
the aptamers are immobilized to one surface of the membrane.

5. The membrane-type surface-stress sensor according to claim 1, wherein
the aptamers are immobilized to both surfaces of the membrane.

6. The membrane-type surface-stress sensor according to claim 1, wherein
the aptamers are immobilized to the membrane via a conjugate of avidin or an avidin derivative and biotin or a biotin derivative.

7. The membrane-type surface-stress sensor according to claim 1, comprising:
a metal membrane on the surface of the membrane, wherein
the aptamers are immobilized to the surface of the membrane via the metal membrane.

8. The membrane-type surface-stress sensor according to claim 1, wherein
the aptamers are immobilized to the membrane surface via a linker.

9. The membrane-type surface-stress sensor according to claim 8, wherein
a length of the linker in each of aptamers is substantially constant.

10. The membrane-type surface-stress sensor according to claim 8, wherein
the linker contains a silane coupling agent.

11. The membrane-type surface-stress sensor according to claim 8, wherein
the linker contains a crosslinking agent.

12. The membrane-type surface-stress sensor according to claim 8, wherein
a main chain length of the linker is 1 to 15.

13. The membrane-type surface-stress sensor according to claim 1, wherein
the aptamers are immobilized to the surface of the membrane via a silane coupling agent.

14. The membrane-type surface-stress sensor according to claim 1, wherein
the sensor substrate has a plurality of support regions, and
the plurality of support regions each supports the membrane.

15. The membrane-type surface-stress sensor according to claim 14, wherein
the plurality of membrane-type surface-stress sensors include sensors having aptamers for different targets immobilized thereon.

16. The membrane-type surface-stress sensor according to claim 1, wherein
the sensor substrate has a circuit,
the support region has a plurality of piezoresistive elements, and
the circuit is a Wheatstone bridge circuit having the plurality of piezoresistive elements.

17. A method for analyzing a target, comprising:
immersing the membrane-type surface-stress sensor according to claim 1 in a sample liquid;
applying a voltage to the membrane-type surface-stress sensor in a liquid phase; and
analyzing a target in the sample liquid by measuring a stress change of the piezoresistive element in the membrane-type surface-stress sensor.

18. The method according to claim 17, wherein
in the voltage-applying, the liquid phase is the sample liquid, and
the voltage-applying is performed directly after the immersing.


SEQUENCE LISTING (2 sequences)

SEQ ID NO: 1 - DNA, 20 bp, Artificial Sequence
  Thrombin aptamer
ggttggtgtggttggttttt

SEQ ID NO: 2 - DNA, 20 bp, Artificial Sequence
  poly T
tttttttttttttttttttt